(19) United States
(12) Patent Application Publication
(10) Pub. No.: US 2012/0150051 A1
(43) Pub. Date: Jun. 14, 2012

(54) BLOOD PRESSURE CUFF

(75) Inventors: Matthew J. Kinsley, Liverpool, NY (US); John A. Lane, Weedsport, NY (US); David E. Quinn, Auburn, NY (US); Tyson B. Whitaker, Arden, NC (US); Raymond A. Lia, Auburn, NY (US); Robert L. Vivenzio, Auburn, NY (US); Henry J. Smith, III, Auburn, NY (US); Jeffrey J. Perkins, Tully, NY (US)

(73) Assignee: WELCH ALLYN, INC., Skaneateles Falls, NY (US)

(21) Appl. No.: 12/963,843

(22) Filed: Dec. 9, 2010

(51) Int. Cl. A61B 5/022

(52) U.S. Cl. 600/499

(57) ABSTRACT

A blood pressure cuff is defined by a first sheet and a second sheet. The first sheet has a first interior-inflatable surface attached to a second interior-inflatable surface of the second sheet to form an interior-inflatable portion between the first sheet and second sheet. The interior-inflatable portion is in communication with an opening to fluidly interconnect the interior-inflatable portion with an exterior of the cuff. A textured surface is included on the second interior-inflatable surface.

### FIELD OF THE APPLICATION

This invention relates generally to the non-invasive measurement of blood pressure and, more particularly, to an improved cuff for a sphygmomanometer and a method of manufacturing the same.

### BACKGROUND OF THE INVENTION

The measurement of blood pressure is a common procedure used in hospitals, clinics and physicians' offices as a tool to assist in diagnosis of illness and monitoring of sick patients, as well as an indicator of the general status of a person's health. In standard non-invasive blood pressure measurement practice, blood pressure is measured using an inflatable sleeve, commonly referred to as a cuff, to measure arterial blood pressure. The cuff, which is adapted to fit around a limb over an artery of a patient, typically around the patient's upper arm over the brachial artery, includes an interior chamber adapted to be inflated with air to provide pressure on the artery.

A detailed discussion and description of an exemplary embodiment of the cuff is presented in U.S. Patent Publication No. 2010/0298725 A1 filed on Feb. 12, 2010, and entitled “Recyclable or Biodegradable Blood Pressure Cuff, the contents of which are relied upon and incorporated herein by reference, U.S. Patent Publication No. 2010/0298724 A1 filed on May 19, 2009, and entitled “Recyclable or Biodegradable Blood Pressure Cuff”; the contents of which are relied upon and incorporated herein by reference, U.S. Patent Publication No. 2007/0185401 A1 filed on Feb. 6, 2016, and entitled “Blood Pressure Measurement”, the contents of which are relied upon and incorporated herein by reference, commonly owned U.S. Pat. No. 7,429,245 issued on Sep. 30, 2008, and entitled “Motion Management In a Fast Blood Pressure Measurement Device”, the contents of which are relied upon and incorporated herein by reference, and commonly owned U.S. Pat. No. 6,036,718 issued on Mar. 14, 2000, and entitled “Bladderless Blood Pressure Cuff”, the contents of which are relied upon and incorporated herein by reference.

The measurement of blood pressure is a strong tool in the diagnosis of many medical conditions and diseases, for example heart disease. The measurement of blood pressure is performed as part of a standard physical examination and the blood pressure of seriously ill patients is monitored on a very frequent basis. The blood pressure of a patient is usually determined by the use of a sphygmomanometer. The sphygmomanometer is used by wrapping an inflatable cuff around an arm or leg. The cuff is inflated by a pneumatic bulb that is connected to the cuff. The cuff is inflated to provide a certain amount of pressure on the artery in the arm or leg, typically just enough pressure to restrict the blood flow in a major artery in the arm or leg. The health care provider utilizes a stethoscope to listen for blood flow in the artery while the cuff is slowly deflated. The cuff is deflated by allowing air to slowly flow out of the tube. The health care provider hears the blood flow resume while simultaneously reading a gauge on the sphygmomanometer which has predetermined pressure measurements thereon. The pressure in the cuff is continuously reduced until the health care provider can no longer hear the blood flow. The health care provider thereby determines the systolic and diastolic blood pressure of the patient.

Electronic blood pressure measurement devices for automatically inflating the cuff and automatically sensing the blood pressure either during inflation of the cuff or during deflation of the cuff are well-known in the art. In such devices, a motor driven pump is operatively connected to the interior chamber, or bladder, of the cuff by, for example, means of a tube, often referred to as a lumen. Upon activation of the pump motor, air is pumped by the pump through the tube to inflate the interior chamber of the cuff to a pressure sufficient to stop the blood flow through the artery. A bleed valve is also operatively connected in fluid communication with the interior chamber to permit depressuring of the interior chamber when it is desired to deflate the cuff, either step-wise or rapidly, as desired. Generally, a pressure sensing device, typically a pressure transducer, is operatively connected in fluid communication with the interior chamber of the cuff to directly sense the pressure within the interior chamber of the cuff.

Automated blood pressure measurement devices commonly employ either an ausculatory technique or an oscillometric technique to detect when the systolic blood pressure, which corresponds to the cessation of blood flow through the artery, is reached, and when the diastolic blood pressures, which corresponds to unrestricted blood flow through the artery, is reached. In a conventional ausculatory method, a sound sensing device, commonly a microphone, is provided in operative association with the cuff to listen for pulsating sounds, known as Korotkoff sounds, associated with the flow of blood through an artery under pressure. In a conventional oscillometric approach, one or more pressure sensing devices, for example pressure transducers, are provided in operative association with the cuff to detect small oscillatory pressures that occur within the cuff as the result of the pulsating characteristic of blood flow through the artery.

Electronic circuitry, including a central processing unit, is provided that processes the signals from the cuff pressure sensor, and, if present, the microphone or additional pressure sensors, and determines the systolic and diastolic blood pressures. Typically, a digital display is also provided for displaying the systolic and diastolic blood pressures. The signals indicative of the systolic and diastolic blood pressure measurements may be transmitted to an external device, such as a laptop or a patient monitor, for display and/or data recording.

Automated blood pressure measurement devices may be either two-lumen or single lumen devices. In a two-lumen apparatus, the first lumen provides a conduit connecting the inflation chamber of the cuff in fluid communication with the pump and the second lumen provides a conduit connecting the inflation chamber of the cuff in fluid communication with a pressure transducer, or other pressure sensing device. Therefore, the chamber is inflated during the inflation period by the pump passing air flow through the first lumen, while the pressure within the cuff is monitored independently through a static second lumen, unaffected by the flow of air through the first lumen. In a single lumen device, however, the inflation chamber of the cuff is connected in fluid communication with both the pump and the pressure sensor through the conduit of single lumen. Consequently, on a single lumen device, the pressure sensed by the pressure sensor will be impacted by the pressure losses experienced by the air flowing through the first lumen.

A detailed discussion and description of the operation of an exemplary embodiment of an electronic apparatus for the non-invasive measurement of blood pressure is presented in U.S. Pat. No. 7,429,245, the contents of which are relied upon and incorporated herein by reference.

Prior to the inflation period, the inflation chamber may be subject to the introduction of folds, creases, pinched material, or the like. These deformations of the inflation chamber, and the material comprising the inflation chamber can be caused by, for example, material characteristics, folding of the inflation chamber for storage, creasing of the inflation chamber during packaging, or from heavy objects placed on the inflation chamber while the cuff is not in use. Such deformations, however, have a tendency to create areas of low inflation pressure during an inflation period. Thus, the force of the cuff as applied to the limb of a patient during an inflation period may vary across the surface area of the cuff that is in contact with the limb of a patient.

In practice, deformations within an inflation chamber may cause pillowing or lobeing effects, which are areas of low pressure during an inflation period near a deformation in the inflation chamber. If a pillowing or lobeing effect occurs over or near an artery targeted for occlusion during a blood pressure measurement processes, an operator may unknowingly conduct a measurement without adequately occluding the artery. Further, pillowed or lobed areas of the inflation chamber have a tendency to release, or pop, as the inflation pressure increases during an inflation period. The release of pillowed or lobed areas during an automated blood pressure measurement sequence that measures blood pressure during an inflation period may result in an inaccurate blood pressure reading.

Therefore, there is a need presently to provide a blood pressure cuff having an inflation chamber resistant to the formation of pillowed or lobed areas during an inflation cycle.

### SUMMARY OF THE INVENTION

According to one aspect, there is disclosed a blood pressure cuff having a first sheet and a second sheet. A first interior-inflatable surface of the first sheet is attached to a second interior-inflatable surface of the second sheet to form an interior-inflatable portion between the first sheet and second sheet. The interior-inflatable portion is in communication with an opening to fluidly interconnect the interior-inflatable portion with an exterior of the cuff. A texture is included on the second interior-inflatable surface having a means for reducing artifact noise during an inflation cycle.

In another aspect of the cuff, the texture comprises a row of geometric formations extending in a substantially transverse direction of the cuff from a first longitudinal edge of the second sheet to a second longitudinal edge of the second sheet. Further, each geometric formation is defined by a first pair of grooves and a second pair of grooves in which the grooves provide a plurality of passages for the passage of gases or liquids while the cuff is in the deflated state. The depth of each pair of grooves is about 0.005 inches to 0.012 inches.

According to another aspect of the cuff, the first pair of grooves intersect the second pair of grooves such that a shape of the geometric formation is a parallelogram. The transverse dimension of the parallelogram is about 0.08 inches to 0.25 inches and a longitudinal dimension of the parallelogram is about 0.04 inches to 0.125 inches.

In another exemplary embodiment, the first pair of grooves intersect the second pair of grooves such that a shape of the geometric formation is a hexagon.

In yet another embodiment of the cuff, the inflation texture has a random pattern of geometric formations extending in a substantially transverse direction of the cuff.

According to another aspect of the cuff the first sheet has an interior-inflatable surface having an inflation texture.

In yet another version of the cuff, a blood pressure cuff is provided with a first sheet having an opening and a first interior surface of the first sheet is attached to a second interior surface of the second sheet. A bladder having another opening is inserted between the first sheet and the second sheet such that the opening of the bladder is substantially aligned with the opening of the first sheet to provide a fluid connection between an interior-inflatable portion of the bladder with the exterior of the cuff. Further, the interior-inflatable portion includes a friction-reducing component, which can comprise, for example, one or more of a separator, a powder, liquid, gel, or grease. The co-efficient of friction between the top interior surface of the bladder and the bottom interior surface of the bladder is about 0.7.

In another aspect of the cuff, the friction-reducing component is an inflation texture on an interior surface of the bladder having a row of geometric formations extending in a substantially transverse direction of the cuff. Each of the geometric formations is defined by a first pair of grooves and a second pair of grooves.

In another exemplary embodiment of the present disclosure, a method of manufacturing a blood pressure cuff is disclosed. The method includes providing both a first sheet and a second sheet. An opening is cut into the first sheet while a texture is applied to an interior surface of the second sheet. Further, a first interior surface of the first sheet is attached to a second interior surface of the second sheet.

In such an exemplary embodiment, the method further includes applying the texture to the second interior surface by using either heat forming or embossing techniques. The application process should be such that the second interior surface has at least one row of geometric formations extending in a substantially transverse direction of the cuff from one longitudinal edge of the second sheet to the other longitudinal edge.

In another aspect of the method, the process of attaching the first sheet to the second sheet includes using one of heat sealing, ultrasonic welding, or RF welding processes.

In yet another exemplary embodiment of the present disclosure, another method of manufacturing a blood pressure cuff is provided. The method includes providing both a first sheet and a second sheet and applying a first friction-reducing component to an interior surface of the first sheet and an interior surface of the second sheet. An opening is cut into the first opening into the first sheet and a bladder is inserted between the bottom of the first sheet and the bottom of the second sheet. Another opening is cut into the bladder and a second friction-reducing component is applied to an interior surface of the bladder. The opening of the first sheet and opening of the bladder are substantially aligned, and the first sheet is attached to the second sheet.

In such an exemplary embodiment, the method further includes a process of applying the second friction-reducing component through either heat forming or embossing, such that the second friction-reducing component comprises a row of geometric formations extending in a substantially transverse direction of the cuff, and wherein each geometric formation is defined by two pairs of grooves.

In yet another exemplary embodiment of the present disclosure, there is disclosed a blood pressure cuff having a first sheet and a second sheet. A first interior-inflatable surface of the first sheet is attached to a second interior-inflatable surface of the second sheet to form an interior-inflatable portion between the first sheet and second sheet. The interior-inflatable portion is in communication with an opening to fluidly interconnect the interior-inflatable portion with an exterior of the cuff. The interior-inflatable portion includes a friction-reducing component.

These and other features and advantages will become readily apparent from the following Detailed Description, which should be read in conjunction with the accompanying drawings.

### BRIEF DESCRIPTION OF THE DRAWINGS

FIG. 1 is a partially exploded view of a disposable blood pressure cuff according to a first embodiment;

FIG. 2 is a partially assembled view of the disposable blood pressure cuff of FIG. 1;

FIG. 3 is a top perspective view of the disposable blood pressure cuff of FIGS. 1 and 2;

FIG. 4a is a perspective view of a disposable blood pressure cuff made in accordance with another embodiment;

FIG. 4b is a perspective view of a disposable blood pressure cuff made in accordance with yet another embodiment;

FIG. 4c is an exploded view of the disposable blood pressure cuff of FIG. 8b;

FIG. 4d is a top view of an unfolded disposable blood pressure cuff made in accordance with yet another embodiment;

FIG. 4e is a top view of the disposable blood pressure cuff of FIG. 4d in an assembled state;

FIG. 4f is a top view of an unfolded disposable blood pressure cuff made in accordance with yet another embodiment;

FIG. 4g is a top view of the disposable blood pressure cuff of FIG. 4f in an assembled state;

FIG. 5a is an enlarged portion of an embodiment of an interior-inflatable surface of a second sheet of an interior-inflatable surface of a second sheet;

FIG. 5b is an enlarged portion of another embodiment of an interior-inflatable surface of a second sheet;

FIG. 5c is an enlarged portion of yet another embodiment of an interior-inflatable surface of a second sheet;

FIG. 5d is an enlarged portion of yet another embodiment an interior-inflatable surface of a second sheet;

FIG. 6a is a partial cross-sectional view along line 6-6 in FIG. 4b having second sheet 155 formed by embossing; and

FIG. 6b is a partial cross-sectional view along line 6-6 in FIG. 4b having second sheet 155 formed by heat forming.

### DETAILED DESCRIPTION

The following description relates to several exemplary embodiments of a blood pressure cuff or sleeve, as well as related methods for manufacturing the sleeves. It will be readily apparent, however, that a number of other variations and modifications embodying the inventive concepts described herein are possible. In addition, certain terms such as, “top”, “bottom”, “upper”, “lower”, “above”, “below”, “over”, “beneath”, “left”, “right”, and the like are used throughout in order to provide a suitable frame of reference with regard to the accompanying drawings. These terms, however, are not intended to be overlimiting, except where so specifically noted.

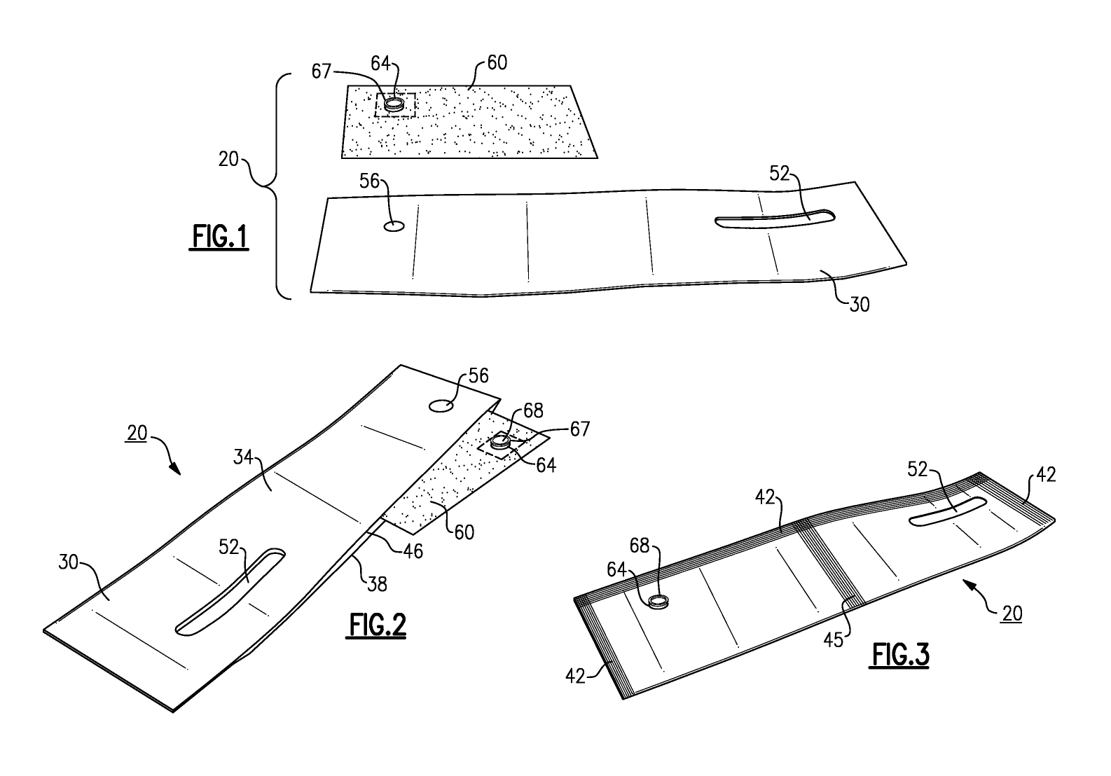

Referring to FIGS. 1-3, there is shown a blood pressure cuff 20 that is made in accordance with a first embodiment. The cuff 20 is defined by a highly flexible sleeve member 30, which according to this exemplary embodiment is made from a thin material, such as paper or nylon, or a durable, flexible and fluid-impermeable material such as polyethylene, polyester, polyvinylchloride, or polypropylene. The envelope-like structure of the flexible sleeve member 30, according to this exemplary version, is made up of a single sheet of material that is folded along one edge to define a pair of planar sleeve portions, or sheets, 34, 38, each sleeve portion having a length dimension that is significantly larger than a corresponding width dimension. The envelope-like structure is created by sealing the remaining edges 42, FIG. 3, of the sleeve 30 by bonding, heat sealing, ultrasonic or RF welding or other suitable means. Alternatively, the cuff 20 can be made from multiple sheets and sealed along all peripheral edges thereof. A slotted region 52 is formed through each of the sleeve portions 34, 38 on one side of the flexible sleeve 30, while a circular opening 56 is provided through an opposite side thereof through one of the planar sheets 34.

According to this exemplary version, an inflatable bladder 60, shown in FIGS. 1, 2, is placed within the interior 46 of the side of the sleeve 30 having the circular opening 56 prior to sealing of the peripheral edge 42, FIG. 3. Alternatively, an inflatable portion of the flexible sleeve member 30 can be sealed on all edges, including an interior bordering edge 45, FIG. 3, thereof as described in a later embodiment. An exterior port is provided herein in the form of a socket 64 that extends from the bladder 60 and communicates fluidly with the interior thereof. The socket 64 is defined herein as an open-ended cylindrical cavity having a circumferential lip 67, which according to this embodiment extends from the exterior of the bladder 60 and is sized to extend through the circular opening 56 of the sleeve member 30. It will be readily apparent that the opening 56 can assume other shapes depending on the socket or port that is used therewith; for example, a hexagonal or other suitably shaped opening and port could be utilized. An opening 68 within the socket 64 extends into the interior inflation chamber, or interior-inflation portion, of the bladder 60 (not illustrated). As a result, the socket 64 enables fluid as well as mechanical interconnection with blood pressure measuring apparatus including a gage housing or hose adapter by way of a releasable snap-fitting connection. Additional details concerning a suitable socket design and socket connectors (not illustrated) are provided in U.S. Pat. No. 6,422,086, and U.S. Patent Publication No. 2006/0263600 A1, for which the entire contents of both are herein incorporated by reference.

The bladder 60 according to this embodiment is defined by a fluid-impermeable and flexible material and is further defined by a substantially rectangular configuration. It should be noted, however, that this shape should not be limiting, meaning other suitable geometries can be easily utilized. The bladder 60 can be inflated, as described in greater detail below by pneumatic means, such as a pump or bulb (not shown) attached preferably in releasable fashion to the socket 64.

Prior to either the assembly or inflation of the bladder 60, a friction-reducing component (not illustrated) may be added to the interior and/or exterior of the inflation chamber of the bladder 60. The friction-reducing component can be in the form of an additive such as, for example, a powder, a liquid, a gel, grease, or the like. The addition of such a friction-reducing component acts to reduce the coefficient of friction between a top interior surface (not illustrated) and a bottom interior surface (not illustrated) of the bladder 60 and/or the coefficient of friction between the bladder 60 and one or both of the interior surfaces (not illustrated) of sleeve portions 34, 38.

Additionally, these types of friction-reducing components may also act to create a separation between a top interior surface and a bottom interior surface of the bladder 60 in a deflated state to allow, for example, the flow of gases, liquids, or the like. Further, an inflation texture, as described in more detail below, may be added to at least a portion of the interior-inflatable portion of the bladder 60 to create a separation between a top interior surface and a bottom interior surface of the bladder 60 in a deflated state.

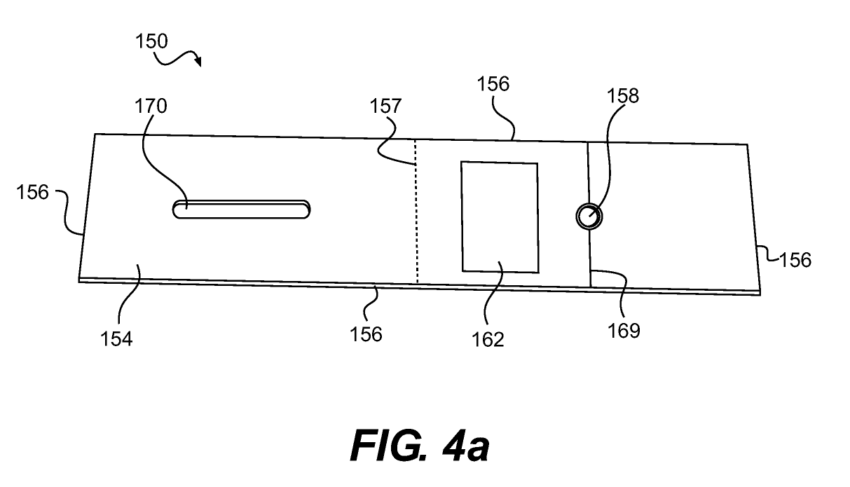

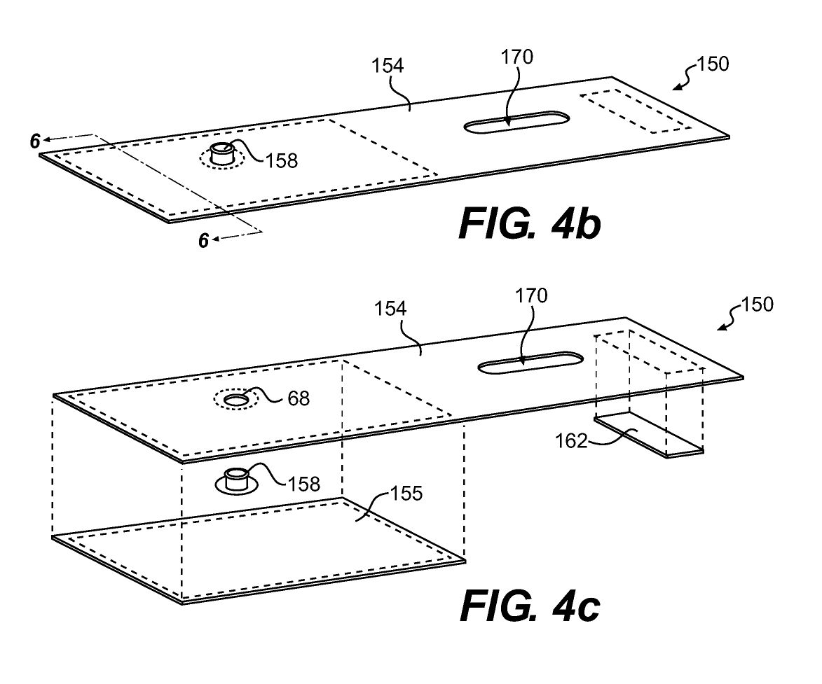

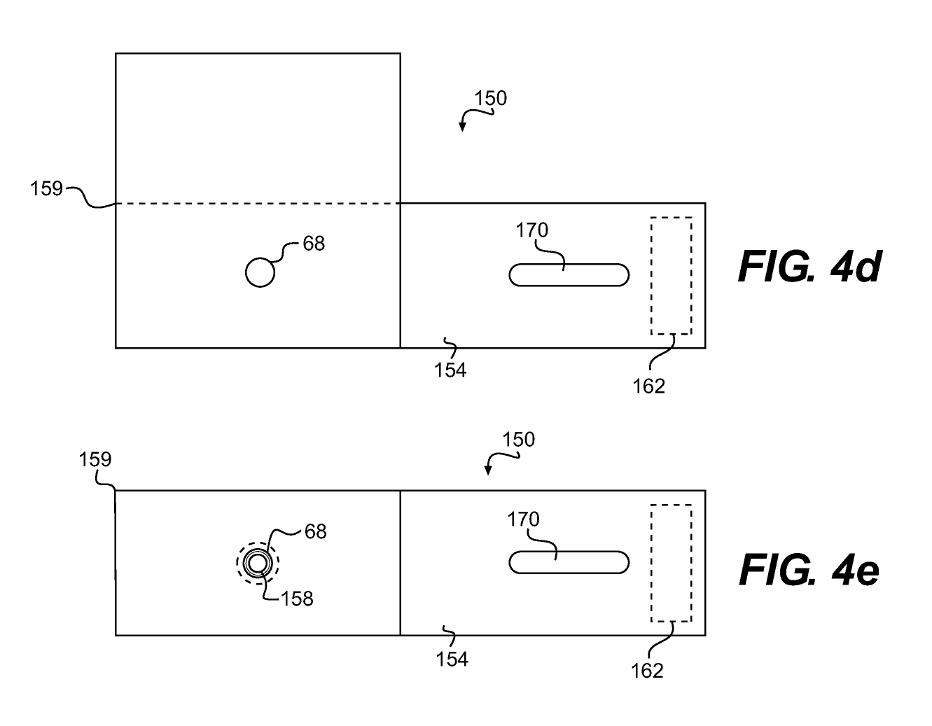

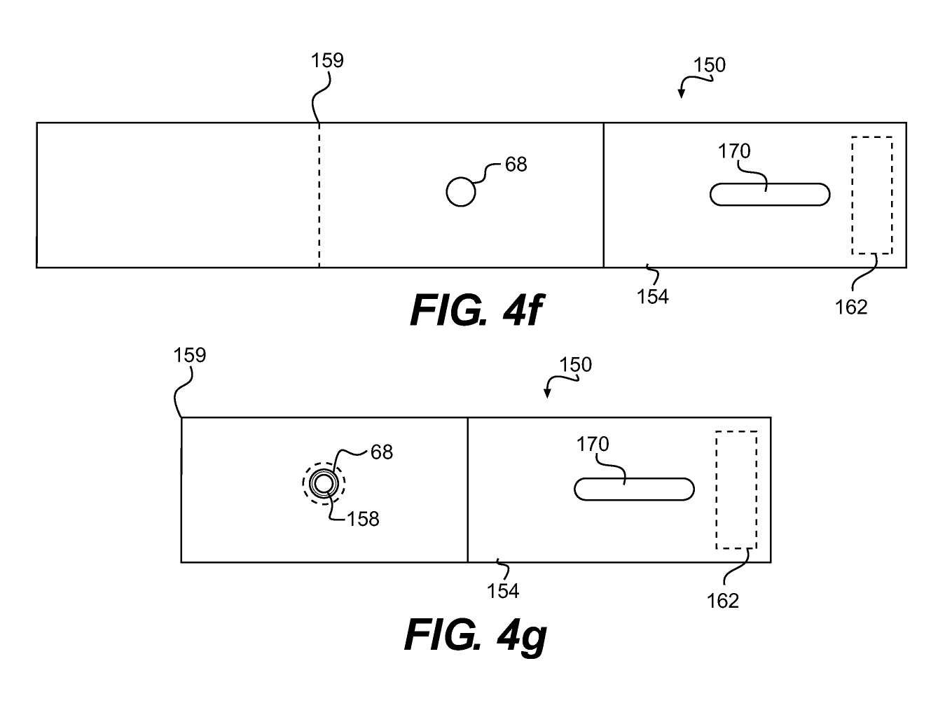

The slotted portion 52, as noted above, extends through each of the sleeve portions 34, 38 and along the major dimension of the sleeve member 30 opposite that of the bladder 60. The major dimension of the slotted portion 52 is aligned with the port 56 and is configured, as shown in FIG. 4, to provide a means of limiting the degree of wrapping of the cuff 20 about the limb of a patient (not shown) when the sheets are looped. As a result, the range of arm sizes that the cuff can be used is limited. The sleeve 30 can be retained in a wrapped form, such as by means of an adhesive, hook and loop fasteners, clips or other suitable attachment means.

Referring to FIGS. 4a-g, there is disclosed a blood pressure cuff 150 that is made in accordance with another exemplary embodiment. The cuff 150 according to this embodiment is made from at least one or a pair of planar sheets made from a highly flexible material, each sheet having a first side that is fluid impermeable. According to the present embodiment, a pair of sheets 154 consisting of a fluid impermeable side made from polypropylene and, optionally, a nonwoven side made from polyethylene that are sealed together to form a single sheet. One or two sheets 154 are used wherein the fluid impermeable sides (not shown) of each sheet are placed into adjacent relation to create a cuff interior and the nonwoven sides form an exterior in which all of the peripheral edges 156 are sealed by appropriate means, such as, for example, bonding, heat sealing, ultrasonic treatment, or RF welding.

Alternatively, the entire cuff can be made from a single material, such as polypropylene, enabling recyclability. Referring to the embodiment in FIGS. 4b and 4c, sheet 154, which includes slotted portion 170, preferably comprises a film on the surface of sheet 154 including the hook fastener portion 162 and a non-woven fabric laminated to the film forming a top surface of the sheet 154. The film in this embodiment has a thickness of about 0.002 to 0.008 inches, and preferably 0.0025 to 0.0055 inches, and a weight of about 1.0 to 7.0 ounces per square yard, and preferably 1.2 to 4.6 ounces per square yard. Additionally, the non-woven fabric of this embodiment has a weight of about 0.5 to 3.0 ounces per square yard, and preferably 0.9 to 1.4 ounces per square yard. The resulting laminated fabric of this embodiment will have a resulting weight of about 1.5 to 15.0 ounces per square yard, and preferably about 2.1 to 6.0 ounces per square yard. A creep elongation of the laminated fabric, as measured with a one inch by one inch test sample loaded with five pounds over six minutes, should be less than about 0.0065 inches, and preferably less than about 0.0022 inches.

In the embodiment of FIG. 4a, an intermediate transverse seal 157 is also formed at or near the middle of the length dimension of the cuff 150, thereby dividing the cuff into two adjacent sections, one of which is capable of inflation as described herein. An opening is formed in one of the planar sheets 154 in one of the sections wherein a port supported upon a smaller sheet section (not shown), preferably made from the same material as the sheets 154 is bonded to the interior of the sheet, and in which the port herein is defined by a socket 158 that extends through the opening wherein a fluid tight seal is created about the periphery of the socket within the opening. The socket 158 includes a relatively flexible circumferential or annular lip and is also preferably formed from the same material (i.e., polypropylene) as those constituting the sleeve sheets 154. A slotted portion 170 is formed in the opposite side of the cuff 150 wherein the major dimension of the slotted portion is aligned with the extending socket 158. According to this embodiment, the slotted portion 170 and opening/socket are provided at substantially the center of the width dimension of the cuff 150.

Adjacent the opening and extending socket 158, a hook fastener portion 162 is provided on the exterior of the cuff 150 on one side thereof. In this embodiment, the material of the cuff on the non-woven exterior of the sheets 154 is defined by a micro-structure that creates adhesion with the hook fastener portion 162 when the cuff 150 is wrapped around the limb of a patient. Due to the nature of this material, there is no need to provide a separate loop fastener portion to act as closure means for the cuff 150.

When wrapped, the slotted portion 170 is sized to accommodate the socket 158 and the slotted portion is further sized to be wrapped only within a predetermined range of limb (arm or leg) circumferences. The slotted portion 170 in combination with the extending socket 158 serves numerous functions. First, and as noted, the slotted portion 170 will only accommodate a predetermined range of arm circumferences, in which the slotted portion can be formed to accommodate a class of patient (e.g., a child, an adult, a large adult, etc). In addition, the slotted portion 170 serves as a guide to wrapping the cuff 150 about the limb 180 given that the socket 158 must be fitted within the slotted portion when wrapped. Still further, the use of a slotted portion 170 and socket 158 permits the port to be flexibly located on the cuff 150 without interfering with the attachment, such as those involving hook and loop fasteners. In fact, the hook fastener portion 162 can also be positioned more conveniently along the exterior of the cuff 150 than in previously known cuffs. In this embodiment, the hook fastener portion 162 can be positioned more inboard (that is, inboard relative to the nearest lateral edge 156 of the cuff) such that attachment can occur within the overlapping portion of the cuff 150 that includes the slotted portion 170. By moving the attachment (fastener) portions more inboard, greater adhesion is achieved using less total surface area. Moreover and by selection of materials as in this embodiment, manufacture is simplified in that a separate loop fastener portion is not required given the inherent adhesive quality of the exterior sleeve material.

In this version, the durability of the material is affected with each attachment and subsequent removal of the cuff 150 from the hook fastener portion 162. This degradation of material influences the ability of the material to further adhere in those areas, thereby rendering the cuff 150 incapable of attachment after a finite number of uses. Depending on the material, this finite number of uses could be made to be one or several.

FIGS. 4b and 4c illustrate another embodiment of the cuff 150 having a bladder sheet, or second sheet, 155 with a length shorter than the length of both the sheet 154 and the cuff 150. The cuff 150 according to this embodiment is made from sealing the bladder sheet 155 to the sheet 154 in at least a region around the opening 68 to form a single sheet. In this configuration, an interior-inflatable portion is created between sheet 154 and bladder sheet 155 with the opening 68 fluidly interconnecting the interior-inflatable portion with the exterior of the cuff 150. A socket 158, as described above, extends through the opening 68 wherein a fluid tight seal is created about the periphery of the socket within the opening. The slotted portion 170 and opening/socket 68/158 are provided at substantially the center of the width dimension of the cuff 150, while the hook fastener portion 162 is also provided on sheet 154 on the side including bladder sheet 155.

In the embodiment in FIGS. 4d and 4e of the cuff 150, sheet 154 comprises a substantially L-shaped configuration generally having a lateral area, a central area, and a vertical area. The lateral area includes slotted portion 170, and hook fastener portion 162, while the central area includes the opening 68. The cuff 150 is formed with this embodiment by folding the vertical area under the central area along folded portion 159 of the sheet 154. A socket 158 is also extended through opening 68 and attached to sheet 154 as described above. A bladder is then formed by sealing all peripheral edges between the central and vertical areas, except for areas around folded portion 159, which already form a fluid impermeable barrier. The slotted portion 170 and opening/socket 68/158 are provided at substantially the center of the width dimension of the cuff 150, while hook fastener portion 162 is also provided on sheet 154 on the surface where the vertical area is attached to the central area of the sheet 154.

FIGS. 4f and 4g illustrate an additional embodiment of the cuff 150, in which sheet 154 comprises a substantially rectangular configuration generally having a left-lateral area, a central area, and a right-lateral area. The right-lateral area includes slotted portion 170, and hook fastener portion 162, while the central area includes the opening 68. Forming the cuff of this embodiment includes folding the left-lateral area under the central area along folded portion 159 of the sheet 154. A socket 158 is also extended through opening 68 and attached to sheet 154 as described above. Sealing all peripheral edges between the central and left-lateral areas, except for areas around folded portion 159, acts to form a bladder. The slotted portion 170 and opening/socket 68/158 are provided at substantially the center of the width dimension of the cuff 150, while hook fastener portion 162 is also provided on sheet 154 on the surface where the left-lateral area is attached to the central area of the sheet 154.

Another embodiment of the cuff 150 contemplates incorporating the cuff 150 in to a single patient or disposable hospital gown (not illustrated). In this embodiment, the cuff is attached to, or integral with, an exterior surface of a sleeve of the gown. Preferably, the cuff 150 is positioned on the sleeve in the area where a patient's upper arm is likely to reside within the sleeve, thereby facilitating wrapping and attaching the cuff 150 around the upper arm of the patient. Thus, taking a blood pressure measurement from the patient does not involve removal of the gown or significant manipulation of the sleeve, which facilitates the measurement.

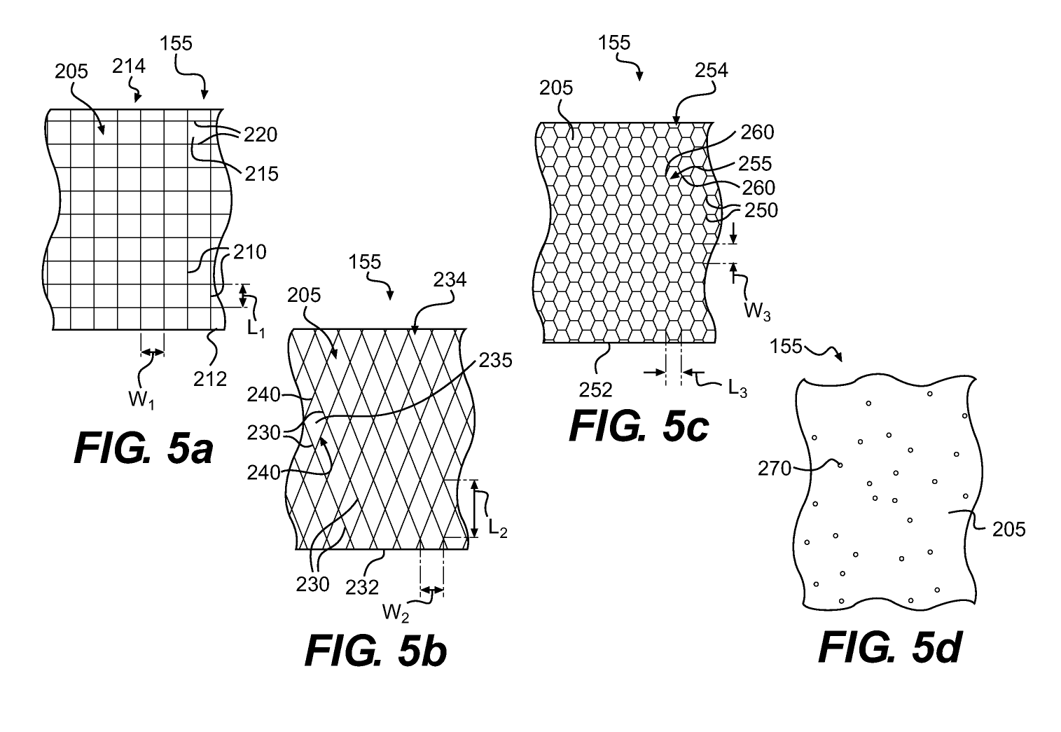

Referring to FIGS. 5a-5d, illustrated are various embodiments of a textured surface 205 formed on a second interior-inflatable surface of the second sheet 155. In FIGS. 5a-5c, the textured surface 205 comprises numerous geometric formations 215, 235, 255 each formed and separated from each other by a first pair of grooves 210, 230, 250 and a second pair of grooves 220, 240, 260, which are both described in more detail below. The geometric formations 215, 235, 255 can take the shape of squares, hexagons, diamonds, parallelograms, or any other suitable shape. The textured surface 205 substantially covers the portion of the second sheet comprising the second interior-inflatable surface. Thus, numerous series of the first pair of grooves 210, 230, 250 substantially extend along the entire longitudinal direction of the second interior-inflatable surface. Similarly, numerous series of the second pair of grooves 220, 240, 260 substantially extend along the entire transverse direction of the second interior-inflatable surface from a first longitudinal edge 212, 232, 252 to a second longitudinal edge 214, 234, 254.

In FIG. 5a, the geometric formations 215 substantially take the shape of a square or a rectangle. Each square/rectangle is formed by a first pair of grooves 210 and a second pair of grooves 220. In one embodiment, the transverse dimension of each complete geometric formation 215, or length L₁, is about 0.08 inches to 0.25 inches. A longitudinal dimension of each complete geometric formation, or width W₁, is about 0.04 inches to 0.125 inches.

The geometric formations 235 of FIG. 5b substantially take the shape of a diamond. The diamonds are formed by a first pair of grooves 230 and a second pair of grooves 240. In the embodiment illustrated, the transverse dimension of each complete geometric formation 235, or length L₂, is about 0.08 inches to 0.25 inches. A longitudinal dimension of each complete geometric formation, or width W₂, is about 0.04 inches to 0.125 inches.

FIG. 5c illustrates the geometric formations 255 that substantially take the shape of a hexagon. The individual hexagons are formed by a first pair of grooves 250 and a second pair of grooves 260. In one embodiment, the transverse dimension of each complete geometric formation 255, or length L₃, is about 0.08 inches to 0.25 inches. A longitudinal dimension of each complete geometric formation, or width W₃, is about 0.04 inches to 0.125 inches.

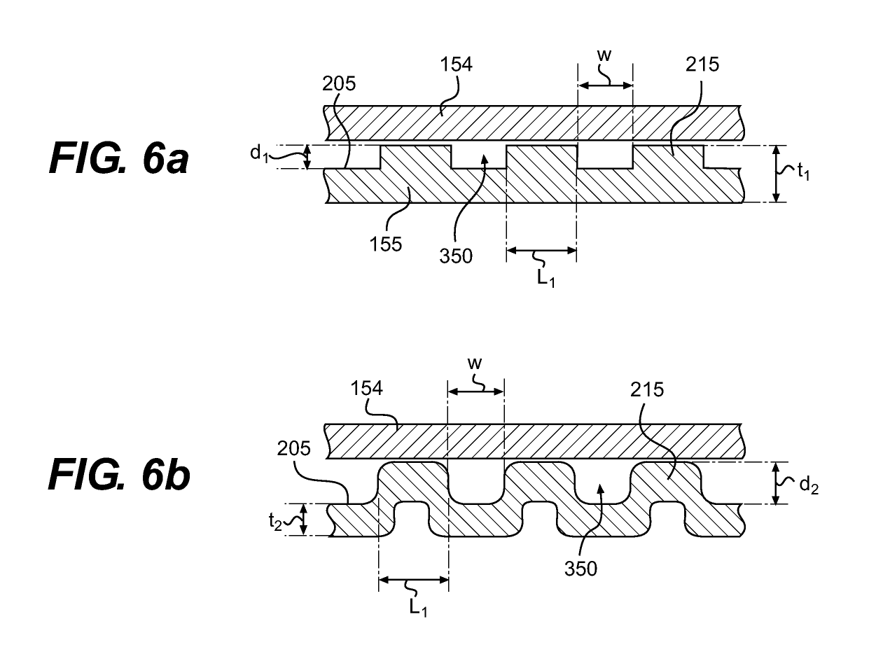

Formation of the grooves illustrated in FIGS. 5a-5c can be achieved in a variety of ways, such as, for example, tool cutting, embossing, heat forming, chemical removal, laser ablation, die formation, or any other suitable material removal or formation process. Referring to FIG. 6A, there is shown a transverse cut-away of the cuff 10 along the line 6-6 indicated in FIG. 4b with the cuff 150 in a substantially deflated state. FIG. 6a illustrates a textured surface 205 of the second interior-inflatable surface of the second sheet 155, of the embodiment in FIG. 5a, produced using a heat forming process. The second interior-inflatable surface second sheet 155 in FIG. 6a is formed by, for example, heating the second sheet 155 to a formable temperature and using a textured roller to form a texture on at least a portion of the second interior-inflatable surface of the second sheet 155. Through the heat forming process grooves 350 are formed. As the series of transverse first pair of grooves 210 and the series of longitudinal second pair of grooves 220 are formed in the second sheet 155, geometric formations 215 are delineated. In the embodiment of FIG. 5a, the second sheet has a thickness, t₁, of thickness of about 0.002 to 0.008 inches, and preferably 0.0025 to 0.0055 inches. The transverse dimension of each complete geometric formation 215, or length L₁, is about 0.08 inches to 0.25 inches, and a depth, d₁, of about half the thickness, t₁, of the second sheet 155. The width, W, of each groove is about 0.002 inches to 0.02 inches.

FIG. 6b illustrates a textured surface 205 of the second interior-inflatable surface of the second sheet 155, of the embodiment in FIG. 5a, produced using an embossing process. Embossed films are available from, for example, Bloomer Plastics, Inc., of Bloomer Wis. The texture of the second interior-inflatable surface second sheet 155 in FIG. 6b is formed by, for example, using a heated form tool or roller to shape the second sheet 155 to have a relatively consistent thickness. Through the embossing process grooves 350 are formed. As series of transverse first pair of grooves 210 and longitudinal second pair of grooves 220 grooves 350 are formed in the second sheet 155, geometric formations 215 are delineated. In the embodiment of FIG. 5a, the second sheet has a thickness, t₁, of about 0.002 to 0.008 inches, and preferably 0.0025 to 0.0055 inches. The transverse dimension of each complete geometric formation 215, or length L₁, is about 0.08 inches to 0.25 inches, and a depth, d₂, of about half the thickness, t₁, of the second sheet 155. The width, W, of each groove is about 0.08 inches to 0.25 inches.

In FIG. 5d the textured surface 205 includes blocks 270 that are substantially circular in form but may take any shape so long as a passage or groove exists between any surrounding blocks 270. The formation of blocks 270 on the textured surface can be in any organized pattern or in a random pattern so long as blocks 270 substantially extend the transverse direction of the second interior-inflatable surface of the second sheet 155.

As discussed above, prior to the inflation period, the bladder 60 or interior-inflatable portion may be subject to the introduction of deformations such as, for example, folds, creases, pinched material, or the like. In practice, deformations within an interior-inflatable portion may cause pillowing or lobeing effects, which are areas of low pressure during an inflation period near a deformation in the inflation chamber. If a pillowing or lobeing effect occurs over or near an artery targeted for occlusion during a blood pressure measurement processes, an operator may unknowingly conduct a measurement without adequately occluding the artery. Further, pillowed or lobed areas of the inflation chamber have a tendency to release, or pop, as the inflation pressure increases during an inflation period. The release of pillowed or lobed areas during an automated blood pressure measurement sequence that measures blood pressure during an inflation period is likely to create an artifact that creates noise in the data sent to a transducer monitoring the pressure inside the interior-inflatable portion. This noise artifact may result in an inaccurate blood pressure reading.

By providing an interior-inflatable portion having a textured surface on an interior-inflatable surface of one or more of the first sheet 154 and the second sheet 155, gases or liquids used to inflate the cuff 150 can bypass or navigate through pillowing or lobeing effects within deformations present in a cuff 150. The interior-inflatable portion of the cuff 150 is therefore allowed to inflate more evenly to remove any deformations that are present. Accordingly, a cuff 150 having a textured surface 205 on an interior-inflatable surface can help to reduce noise artifacts during an inflation cycle and help ensure that the measured artery is adequately occluded.

### PARTS LIST FOR FIGS. 1-6

20 cuff, recyclable or biodegradable
30 sleeve member, flexible
34 planar sheet
38 planar sheet
42 peripheral edges
45 seal, intermediate
46 interior
52 slotted region
56 opening
60 bladder
64 socket
67 circumferential lip
68 opening
150 recyclable blood pressure cuff
154 sheets, planar
155 bladder sheet
156 peripheral edge
157 transverse seal, edge
158 socket
159 fold portion
162 hook fastener portion
169 artery marker
170 slotted portion
205 textured surface
210 first pair of grooves
212 first longitudinal edge
214 second longitudinal edge
215 geometric formation
220 second pair of grooves
230 first pair of grooves
232 first longitudinal edge
234 second longitudinal edge
235 geometric formation
240 second pair of grooves
250 first pair of grooves
252 first longitudinal edge
254 second longitudinal edge
255 geometric formation
260 second pair of grooves
270 block
350 groove
W₁ width
W₂ width
W₃ width
W width
L₁ length
L₂ length
L₃ length
d₁ depth
d₂ depth
t₁ thickness
t₂ thickness

As described above and in the associated drawings, therefore, it will be apparent to those skilled in the art that various modifications and variations can be made in the apparatus and methods of the present invention without departing from the spirit and scope of the invention. Thus, it is intended that the present invention cover the modifications and variations of this invention, provided they come within the scope of the appended claims and their equivalents. In this context, equivalents mean each and every implementation for carrying out the functions recited in the claims, even if those particular functions are not explicitly described therein. Although specific terms are employed herein, they are used in a generic and descriptive sense only and not for purposes of limitation.

What is claimed is:

1. A blood pressure cuff, the cuff comprising:
a first sheet;
a second sheet, wherein a second interior-inflatable surface of the second sheet is attached to a first interior-inflatable surface of the first sheet,
wherein the second interior-inflatable surface has a textured surface; and
an interior-inflatable portion between the first interior-inflatable surface and the second interior-inflatable surface,
wherein the interior-inflatable portion is in communication with an opening to fluidly interconnect the interior-inflatable portion with an exterior of the cuff.

2. The cuff as recited in claim 1, wherein the textured surface comprises a row of geometric formations extending in a substantially transverse direction of the cuff from a first longitudinal edge of the second sheet to a second longitudinal edge of the second sheet.

3. The cuff as recited in claim 2, wherein each geometric formation is defined by a first pair of grooves and a second pair of grooves.

4. The cuff as recited in claim 3, wherein the grooves provide a plurality of passages while the cuff is in the deflated state and permit the passage of a gas.

5. The cuff as recited in claim 3, wherein the first pair of grooves and the second pair of grooves have a depth of about 0.005 inches to 0.012 inches.

6. The cuff as recited in claim 3, wherein the first pair of grooves intersect the second pair of grooves such that a shape of the geometric formation is a parallelogram.

7. The cuff as recited in claim 6, wherein a transverse dimension of the parallelogram is about 0.08 inches to 0.25 inches and a longitudinal dimension of the parallelogram is about 0.04 inches to 0.125 inches.

8. The cuff as recited in claim 3, wherein the first pair of grooves intersect the second pair of grooves such that a shape of the geometric formation is a hexagon.

9. The cuff as recited in claim 1, wherein the textured surface comprises a random pattern of blocks extending in a substantially transverse direction of the cuff.

10. The cuff as recited in claim 1, wherein the first sheet has an interior-inflatable surface having a textured surface.

11. The cuff as recited in claim 1, wherein the textured surface comprises a means for reducing artifact noise during an inflation cycle.

12. A blood pressure cuff, the cuff comprising:
a first sheet with a first opening;
a second sheet,
wherein a second interior surface of the second sheet is attached to a first interior surface of the first sheet; and
a bladder between the first sheet and the second sheet,
wherein the bladder has a second opening substantially aligned with the first opening to fluidly interconnect an interior-inflatable portion of the bladder with an exterior of the cuff, and
wherein the interior-inflatable portion includes a friction-reducing component.

13. The cuff of claim 12, wherein the friction-reducing component is a textured surface on an interior surface of the bladder.

14. The cuff of claim 13, wherein the inflation texture comprises a row of geometric formations extending in a substantially transverse direction of the cuff.

15. The cuff as recited in claim 14, wherein each geometric formation is defined by a first pair of grooves and a second pair of grooves.

16. The cuff of claim 15, wherein a co-efficient of friction between a top interior surface of the bladder and a bottom interior surface of the bladder is less than 0.7.

17. The cuff of claim 12, wherein the friction-reducing component is selected from the group consisting of a separator, a powder, a liquid, a gel, or a grease.

18. A method for forming a blood pressure cuff, comprising:
providing a first sheet and a second sheet;
cutting an opening into the first sheet;
applying a texture to a second interior surface of the second sheet; and
attaching the second interior surface to a first interior surface of the first sheet.

19. The method of claim 18, wherein a process of applying the texture to the second interior surface is selected from the group consisting of heat forming and embossing,
wherein the texture comprises a row of geometric formations extending in a substantially transverse direction of the cuff from a first longitudinal edge of the second sheet to a second longitudinal edge of the second sheet,
wherein each block is defined by a first pair of grooves and a second pair of grooves, and
wherein the first pair of grooves and the second pair of grooves have a depth of about 0.005 inches to 0.012 inches.

20. The method of claim 18, wherein a process of attaching the first interior surface to the second interior surface is selected from the group consisting of heat sealing, ultrasonic welding, and RF welding.

21. A method for forming a blood pressure cuff, comprising:
providing a first sheet and a second sheet;
applying a first friction-reducing component to an interior surface of the first sheet and an interior surface of the second sheet;
cutting a first opening into the first sheet;
providing a bladder;
cutting a second opening into the bladder;
applying a second friction-reducing component to an interior surface of the bladder;
placing the bladder between the interior surface of the first sheet and the interior surface of the second sheet;
aligning the first opening with the second opening; and
attaching the first sheet to the second sheet.

22. The method of claim 21, wherein a process of applying the second friction-reducing component is selected from the group consisting of heat forming and embossing,
wherein the second friction-reducing component comprises a row of geometric formations extending in a substantially transverse direction of the cuff, and
wherein each block is defined by a first pair of grooves and a second pair of grooves.

23. The method of claim 21, wherein the second friction-reducing component is selected from the group consisting of a separator, a powder, a liquid, a gel, or a grease.

24. A blood pressure cuff, the cuff comprising:
a first sheet;
a second sheet, wherein a second interior-inflatable surface of the second sheet is attached to a first interior-inflatable surface of the first sheet,
wherein the second interior-inflatable surface having a textured surface; and
an interior-inflatable portion between the first interior-inflatable surface and the second interior-inflatable surface,
wherein the interior-inflatable portion is in communication with an opening to fluidly interconnect the interior-inflatable portion with an exterior of the cuff, and
wherein the interior-inflatable portion includes a friction-reducing component.
